(19) United States
(12) Patent Application Publication
(10) Pub. No.: US 2010/0016475 A1
(43) Pub. Date: Jan. 21, 2010

(54) IMIDAZOLE SALTS, METHOD FOR PRODUCING THEM, USE THEREOF AND EPOXY RESINS CONTAINING SAID SALTS

(76) Inventors: Manfred Doering, Woerth (DE); Olaf Lammerschop, Krefeld (DE); Thomas Huver, Duesseldorf (DE); Stefan Kreiling, Huechelheim (DE)

Correspondence Address: HENKEL CORPORATION, One Henkel Way, ROCKY HILL, CT, 06067, US

(21) Appl. No.: 11/942,299

(22) Filed: Nov. 19, 2007

Related U.S. Application Data
(63) Continuation of application No. PCT/EP2006/004160, filed on May 4, 2006.

(30) Foreign Application Priority Data
May 27, 2005 (DE) 10 2005 024 255.3

(51) Int. Cl. C08K 5/3472; C07D 233/56; C08L 63/00

(52) U.S. Cl. 523/461; 548/335.1

(57) ABSTRACT

The invention refers to salts of at least one imidazole of the general formula (I), in which R¹, R², R³ and R⁴ are the same or different and denote hydrogen, an alkyl residue having 1 to 20, preferably 1 to 10, more preferably 1 to 4 carbon atoms, or a substituted or unsubstituted aryl or arylalykl residue having 6 to 10 carbon atoms, and at least one aliphatic or aromatic mono- or dicarboxylic acid. The molar ratio of carboxylic acid to imidazole, based on the functionality of the acid, is 1:1.1 to 1:6. The invention also relates to a method for manufacturing the imidazole salts, to their use, and to epoxy resin compositions containing said salts.

### CROSS-REFERENCE TO RELATED APPLICATIONS

This application is a continuation under 35 USC Sections 365(c) and 120 of International Application No. PCT/EP2006/004160, filed 4 May 2006 and published 7 Dec. 2006 as WO 2006/128542, which claims priority from German Application No. 102005024255.3, filed 27 May 2005, each of which is incorporated herein by reference in its entirety.

### FIELD OF THE INVENTION

The present invention relates to salts of at least one imidazole of the general formula

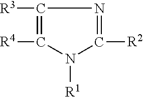

in which R¹, R², R³ and R⁴ are the same or different and denote hydrogen, an alkyl residue having 1 to 20, preferably 1 to 10, more preferably 1 to 4 carbon atoms, or a substituted or unsubstituted aryl or arylalykl residue having 6 to 10 carbon atoms, and at least one aliphatic or aromatic mono- or dicarboxylic acid. The invention further relates to a method for manufacturing said salts, to their use as catalysts in the curing of polyepoxides, and to epoxy resins based on polyepoxides that contain said salts.

### DISCUSSION OF THE RELATED ART

Epoxy resins are aliphatic, cycloaliphatic, or aromatic oligomers that contain oxirane groups and can be crosslinked with resins to yield thermoset plastics. Most epoxy resins are glycidyl ethers of bisphenol A derived from the reaction of bisphenol A with epichlorohydrin. There are also epoxy resins based on epoxidized phenol-formaldehyde or cresol-formaldehyde resins, hydantoin, hexahydrophthalic acid, and the like. The resins can be cured cold with polyfunctional amines, or at high temperature using multifunctional carboxylic acids or carboxylic acid anhydrides. Ester and ether structures are formed as this high-temperature curing proceeds. The possibility also exists of curing epoxy resins by anionic polymerization. Epoxy resins are used for a very wide variety of purposes, for example as adhesives, coatings, for components, large containers, etc. When they are used as engineering structural materials, they are usually reinforced with glass fibers or carbon fibers.

A number of polyepoxides that contain at least two 1,2-epoxy groups per molecule are suitable s epoxies. The epoxy equivalent of these polyepoxides can vary from 150 to 4000. The polyepoxides can in principle be saturated, unsaturated, cyclic or acyclic, aliphatic, alicyclic, aromatic, or heterocyclic polyepoxide compounds. Examples of suitable polyepoxides include the polyglycidyl ethers, which are manufactured by reacting epichlorohydrin or epibromohydrin with a polyphenol in the presence of alkali. Polyphenols suitable for this are, for example, resorcinol, catechol, hydroquinone, bisphenol A (bis-(4-hydroxyphenyl)-2,2-propane), bisphenol F (bis-(4-hydroxyphenyl)methane), (bis-(4-hydroxyphenyl)-1,1-isobutane), 4,4′-dihydroxybenzophenone, bis(4-hydroxyphenyl)-1,1-ethane, and 1,5-hydroxynaphthalene.

Further polyepoxides that are suitable in principle are the polyglycidyl ethers of polyalcohols or diamines. These polyglycidyl ethers are derived from polyalcohols such as ethylene glycol, diethylene glycol, triethylene glycol, 1,2-propylene glycol, 1,4-butylene glycol, triethylene glycol, 1,5-pentanediol, 1,6-hexanediol, or trimethylolpropane.

Further polyepoxides are polyglycidyl esters of polycarboxylic acids, for example reactions of glycidol or epichlorohydrin with aliphatic or aromatic polycarboxylic acids such as oxalic acid, succinic acid, glutaric acid, terephthalic acid, or dimer fatty acid.

Further epoxies are derived from the epoxidation products of olefinically unsaturated cycloaliphatic compounds or of natural oils and fats.

The epoxy resins derived from the reaction of bisphenol A or bisphenol F and epichlorohydrin (DGEBA or DGEBF) are very particularly preferred. Mixtures of liquid and solid epoxy resins are usually used, the liquid epoxy resins by preference being based on bisphenol A and having a sufficiently low molecular weight.

In the manufacture of composite materials, fast cycle times for efficient series production of the fiber-reinforced epoxy-resin components are necessary. These have hitherto been achieved, however, only by means of the prepreg technique, at relatively high temperatures of 140° to 160° C. The use of so-called wet resin techniques eliminates one production step, namely prepregging. Homogeneous resin systems are required, however, and rapid curing is difficult. To achieve good wetting of the fibers or fabric, the viscosity of the resins is lowered by elevating the temperature to 60° C. to 80° C. Crosslinking should not yet be occurring at this temperature. On the other hand, it is desirable to achieve rapid curing by way of a slight temperature increase to 80° C. to 120° C. Even when adhesively bonding metal parts using epoxy resins, it is desirable on the one hand to decrease the viscosity of the resins by temperature elevation, and on the other hand to achieve rapid curing by means of a slight temperature rise.

It is known that the crosslinking of epoxy resins can be accelerated by anionic polymerization at elevated temperature using imidazole salts. These imidazole salts are referred to in the literature in some cases as hardeners and in some cases as catalysts.

U.S. Pat. No. 3,635,894 describes imidazole salts of inorganic acids as catalysts for curing epoxy resins. These are chlorides, bromides, iodides, sulfates, and phosphates.

U.S. Pat. No. 3,642,698 likewise describes imidazole phosphates for the aforesaid purpose.

U.S. Pat. No. 4,331,582 describes imidazole salts of aromatic sulfonic acids as curing catalysts for epoxy resins.

Lastly, U.S. Pat. No. 3,356,645 also describes imidazole salts of organic acids as hardeners or catalysts for curing epoxy resins. Monocarboxylic acids having 1 to 8 carbon atoms, and lactic acid, are recited as organic acids.

In the aforesaid patent documents, imidazole, which can optionally be substituted, is mixed with the acid at a 1:1 molar ratio, or the acid is used at an excess with respect to the imidazole, for manufacture of the imidazole salts.

The known hardeners or catalysts allow the epoxy resins mixed with them to be stored at low temperature, and upon elevation of the temperature, curing of the resins occurs by crosslinking. These known systems are, however, unsatisfactory in one respect: the temperature difference between the temperature at which the resins equipped with the catalyst can be stored without crosslinking, and the temperature at which effective crosslinking occurs, is relatively large.

It is an object of the present invention to reduce this temperature difference and to describe catalysts or hardeners for epoxy resins such that on the one hand no curing occurs at up to approximately 80° C., and on the other hand effective and rapid curing takes place with only a slight temperature elevation to approximately 100° C.

### BRIEF SUMMARY OF THE INVENTION

It has been found, surprisingly, that this object can be achieved by imidazole salts of organic acids that are manufactured with an excess of imidazole. The subject of the present invention is therefore salts of the kind cited initially that are characterized in that the molar ratio of carboxylic acid to imidazole, based on the functionality of the acid, is 1:1.1 to 1:6, by preference 1:2 to 1:4. These base (imidazole)-rich salts act as latent accelerators for epoxy resins, and they are very well suited for rapid processing of epoxy resins. The salts are liquid at room temperature and can easily be mixed with epoxy resins. This mixture can be produced before use, and can heated without difficulty to temperatures of up to approximately 80° C., for example in order to achieve complete wetting of the fibers when manufacturing fiber-reinforced shaped parts. Surprisingly, the residual organic acid ions also positively influence the material properties of the materials, such as glass transition temperature, water absorption, and elasticity. A slight increase in temperature to approximately 100° C. causes a rapid crosslinking to occur, i.e., curing is accelerated by a factor of >2 as compared with the 1:1 salts.

### DETAILED DESCRIPTION OF CERTAIN EMBODIMENTS OF THE INVENTION

Suitable imidazoles are unsubstituted imidazole and alkyl- or aryl-substituted imidazoles. Examples of alkyl-substituted imidazoles are 2-methylimidazole, 2-ethylimidazole, 2-ethyl-4-methylimidazole, 2,4-dimethylimidazole, butylimidazole, 4-butyl-5-ethylimidazole, 2-dodecyl-5-methylimidazole, 2,4,5-trimethylimidazole, 2-undecenylimidazole, 1-vinyl-2-methylimidazole, 2-n-heptadecylimidazole, 2-undecylimidazole, 2-heptadecylimidazole, 1-propyl-2-methylimidazole, 1-cyanoethyl-2-methylimidazole, 1-cyanoethyl-2-ethyl-4-methylimidazole, 1-cyanoethyl-2-undecylimidazole, 1-cyanoethyl-2-phenylimidazole, 1-guanaminoethyl-2-methylimidazole.

Suitable aryl-substituted imidazoles are phenylimidazole, 2,5-diphenylimidazole, 2-phenylethylimidazole, 2-benzylimidazole, 2-methyl-4,5-diphenylimidazole, 2,3,5-triphenylimidazole, 2-styrylimidazole, 1-(dodecyl benzyl)-2-methylimidazole, 2-(2-hydroxy-4-t-butylphenyl)-4,5-diphenylimidazole), 2-(3-hydroxyphenyl)-4,5-diphenylimidazole, 2-p-dimethylaminophenyl)-4,5-diphenylimidazole, 2-(2-hydroxyphenyl)-4,5-diphenylimidazole, 1-benzyl-2-methylimidazole, 2-p-methoxystyrylimidazole.

Preferred imidazoles are unsubstituted imidazole, alkyl-substituted imidazoles having substituents that have 1 to 6 carbon atoms, and aryl-substituted imidazoles having substituents that have 6 to 8 carbon atoms.

The carboxylic acids can be selected from the group made up of substituted or unsubstituted, saturated or unsaturated monocarboxylic acids having 3 to 22 carbon atoms, substituted or unsubstituted, saturated dicarboxylic acids having 2 to 36 carbon atoms, substituted or unsubstituted, unsaturated dicarboxylic acids having 4 to 36 carbon atoms, and substituted or unsubstituted aromatic mono- or dicarboxylic acids.

Particularly to be mentioned as carboxylic acids preferred according to the present invention are: unsaturated substituted or unsubstituted monocarboxylic acids having 3 to 5 carbon atoms and unsaturated substituted or unsubstituted dicarboxylic acids having 4 to 8 carbon atoms, for example, acrylic acid, methacrylic acid, or crotonic acid, fumaric acid, maleic acid, or itaconic acid; saturated substituted or unsubstituted monocarboxylic acids having 1 to 5 carbon atoms and saturated substituted or unsubstituted dicarboxylic acids having 2 to 5 carbon atoms, for example formic acid, acetic acid, propionic acid, pivalic acid, oxalic acid, malonic acid, or succinic acid; saturated or unsaturated, substituted or unsubstituted monocarboxylic acids having 6 to 22 carbon atoms, which can also comprise cycloaliphatic structural elements, for example hexanoic acid, heptanoic acid, cyclohexanecarboxylic acid, 2-ethylhexanoic acid, decanoic acid (C₁₀), myristic acid (C₁₄), palmitic acid (C₁₆), stearic acid (C₁₈), oleic acid, behenic acid (C₂₂); saturated or unsaturated, substituted or unsubstituted dicarboxylic acids having 6 to 36 carbon atoms that comprise, in particular, cycloaliphatic structural elements, for example adipic acid, pimelic acid (C₇), azelaic acid (C₉), sebacic acid (C₁₀), dimer fatty acids having 36 carbon atoms; substituted or unsubstituted aromatic mono- and dicarboxylic acids, for example benzoic acid, phthalic acid, isophthalic acid, terephthalic acid, or naphthalenecarboxylic acids.

The invention also relates to a method for manufacturing the imidazole salts according to the present invention, which method is characterized in that at least one imidazole of the general formula

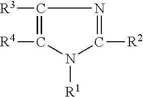

in which R¹, R², R³ and R⁴ are the same or different and denote hydrogen, an alkyl residue having 1 to 20, preferably 1 to 10, more preferably 1 to 4 carbon atoms, or a substituted or unsubstituted aryl or arylalkyl residue having 6 to 12 carbon atoms, and at least one aliphatic or aromatic mono- or dicarboxylic acid, are reacted with one another at a molar ratio of carboxylic acid to imidazole of 1:1.1 to 1:6, by preference 1:2 to 1:4, based on the functionality of the acid, at a temperature between 20° C. and 120° C.

The invention also relates to the use of the imidazole salts as catalysts in the curing of polyepoxides and to epoxy resins based on polyepoxides, having at least two epoxy groups per molecule, that contain the imidazole salts according to the present invention. The proportion of the imidazole salts is advantageously 0.01 to 40 wt %, preferably 1 to 10 wt %, based on the total weight of epoxy resin and salt.

The invention is explained in further detail below with reference to exemplifying embodiments.

### Manufacture of the Imidazole Salts

The imidazole salts were manufactured by reacting the starting materials indicated in the following table, at the molar ratio indicated. For this, the imidazole components were finely powdered and mixed with the acid component with vigorous agitation. Agitation was continued at room temperature for 6 to 12 hours until a homogeneous phase was obtained.

Upon elevation of the temperature to 100° C., the reaction proceeded within 30 to 60 minutes.

The products were obtained as clear, pale-yellow to golden-yellow liquids that in some cases had an oily character.

For comparison, in two experiments 2-ethylhexanoic acid and 1,2-dimethylimidazole and imidazole, respectively were reacted at a 1:1 molar ratio.

### Use of the Imidazole Salts

The imidazole salts were introduced, at a proportion of 5 wt % based on the total weight of the mixture, into an epoxy resin formulation.

### Epoxy Resin Formulation

| Proportion | Description | Manufacturer |
|---|---|---|
| 57% DER 331P | Liquid epoxy resin | Dow Chemical |
| | | Company |
| 10% EPON 164 | Solid epoxy-novolac resin | Resolution |
| 15% PD 3604 | Elastomer-modified epoxy | Struktol |
| | prepolymer (40% NBR*) | |
| 15% PLASTORITE | Mica/quartz/chlorite | Luzenac |
| 3% CAB-O-SIL TS 720 | Pyrogenic silicic acid | Cabot |

*NBR = Nitrile-Butadiene Rubber

### Measuring Tensile Shear Strength

Using an adhesive manufactured in this fashion, cleaned and degreased ZE steel panels of dimensions 100×25 mm (adhesive bonding area 25×10 mm) were adhesively bonded, and cured for 10 minutes at 80° C. and 100° C. The adhesively bonded panels were then investigated in terms of the tensile shear strength of the adhesive bond (ascertained per DIN 53283, “Determination of the adhesive strength of single-lap jointed adhesive bonds” at a rate of 100 mm/min).

At 80° C., the tensile shear strength value for all specimens was 0 MPa, i.e., no curing occurred at this temperature.

At 100° C., the tensile shear strength of the specimens according to the present invention was 1.4 to 8.3 MPa. The comparison specimens displayed tensile shear strength values of 0.1 and 0.4 MPa. The results show that the base-rich imidazole salts according to the present invention cause a considerable acceleration in curing to occur even at 100° C.

| | | Tensile shear strength [MPa] | |
|---|---|---|---|
| | Molar | after 10 min curing at | |
| Composition of the Imidazole Salt | ratio | 80° C. | 100° C. |
| 2-Ethylhexanoic acid:imidazole:N-methylimidazole | 1:1:2 | 0 | 3 |
| 2-Ethylhexanoic acid:imidazole:1,2- | 1:1:2 | 0 | 1.4 |
| dimethylimidazole | | | |
| 2-Ethylhexanoic acid:imidazole:N- | 1:2:1 | 0 | 3.9 |
| methylimidazole | | | |
| 2-Ethylhexanoic acid:1,2-dimethylimidazole | 1:3 | 0 | 8.3 |
| Salicylic acid:imidazole:N-methylimidazole | 1:1:2 | 0 | 3.6 |
| Dodecanoic acid:imidazole:N-methylimidazole | 1:1:2 | 0 | 3.2 |
| Benzoic acid:imidazole:N-methylimidazole | 1:1:2 | 0 | 2.5 |
| Benzoic acid:imidazole:1,2-dimethylimidazole | 1:1:2 | 0 | 3 |
| Succinic acid:imidazole:N-methylimidazole | 1:2:4 | 0 | 1.7 |
| Succinic acid:imidazole:1,2-dimethylimidazole | 1:2:4 | 0 | 5.3 |
| Comparison: | 1:1 | 0 | 0.1 |
| 2-Ethylhexanoic acid:1,2-dimethylimidazole | | | |
| Comparison: | 1:1 | 0 | 0.4 |
| 2-Ethylhexanoic acid:imidazole | | | |

What is claimed is:

1. A salt of at least one imidazole of the general formula 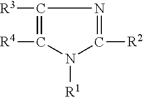
in which R¹, R², R³ and R⁴ are the same or different and denote hydrogen, an alkyl residue having 1 to 20 carbon atoms, or a substituted or unsubstituted aryl or arylalkyl residue having 6 to 10 carbon atoms, and at least one aliphatic or aromatic mono- or dicarboxylic acid, wherein the molar ratio of carboxylic acid to imidazole, based on the functionality of the carboxylic acid, is 1:1.1 to 1:6.

2. A salt according to claim 1, wherein the imidazole is an unsubstituted imidazole, an alkyl-substituted imidazole having one or more substituents having 1 to 6 carbon atoms, or an aryl-substituted imidazole having one or more substituents having 6 to 8 carbon atoms.

3. A salt according to claim 1, wherein the imidazole is selected from the group consisting of imidazole, N-methylimidazole and 1,2-dimethylimidazole.

4. A salt according to claim 1, wherein the carboxylic acid is selected from the group consisting of aliphatic and aromatic mono- and dicarboxylic acids having 1 to 20 carbon atoms.

5. A salt according to claim 1, wherein the carboxylic acid is selected from the group consisting of unsaturated substituted and unsubstituted monocarboxylic acids having 3 to 5 carbon atoms, unsaturated substituted and unsubstituted dicarboxylic acids having 4 to 8 carbon atoms, saturated substituted and unsubstituted monocarboxylic acids having 1 to 5 carbon atoms and saturated substituted and unsubstituted dicarboxylic acids having 2 to 5 carbon atoms,

6. A salt according to claim 1, wherein the carboxylic acid is selected from the group consisting of 2-ethylhexanoic acid, salicylic acid, dodecanoic acid, benzoic acid, and succinic acid.

7. A salt according to claim 1, wherein the molar ratio of carboxylic acid to imidazole, based on the functionality of the carboxylic acid, is 1:2 to 1:4.

8. A method for manufacturing a salt according to claim 1, comprising reacting at least one imidazole of the general formula 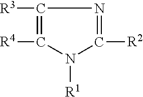
in which R¹, R², R³ and R⁴ are the same or different and denote hydrogen, an alkyl residue having 1 to 20 carbon atoms, or a substituted or unsubstituted aryl or arylalykl residue having 6 to 10 carbon atoms, with at least one aliphatic or aromatic mono- or dicarboxylic acid at a molar ratio of carboxylic acid to imidazole of 1:1.1 to 1:6, based on the functionality of the carboxylic acid, at a temperature between 20° C. and 120° C.

9. The method according to claim 8, wherein the molar ratio of carboxylic acid to imidazole, based on the functionality of the carboxylic acid, is 1:2 to 1:4.

10. A method of curing a polyepoxide, comprising combining said polyepoxide with at least one salt in accordance with claim 1 to form a composition and heating said composition.

11. An epoxy resin composition comprising at least one polyepoxide having at least two epoxy groups per molecule and at least one salt of at least one imidazole of the general formula 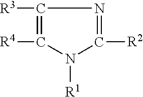
in which R¹, R², R³ and R⁴ are the same or different and denote hydrogen, an alkyl residue having 1 to 20 carbon atoms, or a substituted or unsubstituted aryl or arylalkyl residue having 6 to 10 carbon atoms, and at least one aliphatic or aromatic mono- or dicarboxylic acid, the molar ratio of carboxylic acid to imidazole, based on the functionality of the carboxylic acid, being 1:1.1 to 1:6.

12. An epoxy resin composition according to claim 11, wherein the proportion of the salt is 1 to 10 wt. %, based on the total weight of epoxy resin composition.

13. The epoxy resin composition according to claim 11, wherein the proportion of the salt is 0.01 to 40 wt.%, based on the total weight of epoxy resin composition.
